(19) United States
(12) Patent Application Publication
(10) Pub. No.: US 2024/0148652 A1
(43) Pub. Date: May 9, 2024

(54) A DISPERSIBLE EXTENDED RELEASE COMPOSITION, AND A PROCESS FOR PREPARING THE SAME

(71) Applicant: HERCULES LLC, Wilmington, DE (US)

(72) Inventors: Thomas DURIG, West Chester, PA (US); Kapish KARAN, Wilmington, DE (US); Robert MCCRIMLISK, Sparta, NJ (US)

(73) Assignee: HERCULES LLC, Wilmington, DE (US)

(21) Appl. No.: 17/769,523

(22) PCT Filed: Oct. 16, 2020
(86) PCT No.: PCT/US20/56014

§ 371 Date: Apr. 15, 2022

Related U.S. Application Data
(60) Provisional application No. 62/916,387, filed on Oct. 17, 2019.

(51) Int. Cl. A61K 9/16

(52) U.S. Cl. CPC A61K 9/1641; A61K 9/1652

(57) ABSTRACT

This application discloses a dispersible extended release granule composition comprising: (i) one or more active bio-active ingredients; (ii) at least one water-soluble gel forming polymer; and (iii) at least one water-insoluble lipid material, wherein the bio-active ingredient and the water-soluble gel forming polymer are present in the form of dry, pre-granulated particles either partially or fully coated with the water insoluble lipid material. Also disclosed is a process for preparing such extended release compositions and applications thereof.

### FIELD OF THE INVENTION

The presently disclosed process(es), procedure(s), method(s), product(s), result(s), and/or concept(s) (collectively referred to hereinafter as the “present disclosure”) relates generally to dispersible extended release composition(s), process(s) for the preparation thereof, and application(s) thereof.

### BACKGROUND OF THE INVENTION

Extended release formulations are well known in the field of pharmaceutical and nutritional supplement dosage forms wherein active components/ingredients are released at a predetermined rate slower than the normal rate. Among the most commercially relevant are extended release solid oral dosage forms such as tablets and capsules. A common requirement for solid dosage forms is that tablets or capsules should not be crushed into powder so as not to jeopardize their extended release mechanism, causing catastrophic failure which is typically referred to as “dose dumping” in the industry.

An alternative approach is liquid oral controlled release dosage forms such as syrup suspensions and emulsions. However, liquid oral extended release dosage forms are not widely accepted. One well known example is the Pennkinetic system wherein an active ingredient is loaded onto an ion exchange resin followed by coating of the beadlets with an insoluble polymer such as ethyl cellulose. While effective, such approaches are only suitable for ionic drugs as these systems require chemical binding of drugs to the resin which is not suitable for many drugs and results in relatively low active payloads. Further, liquid oral extended release dosage forms are complex and costly to manufacture, and therefore, are not suitable for active ingredients such as nutritional or dietary supplements wherein the active ingredients need to be delivered in larger gram quantities. Studies demonstrating their benefits typically use the nutraceutical at a high dosage such as 500 mg to 1000 mg. The daily recommended dose of nutraceuticals is generally in the range of 50 mg to 1000 mg per day. Hence, an extended release system that permits, for example, only 20% of the active ingredient by weight would require up to 5 g total dosage form. Such dosage forms either result in big capsules, which are unacceptable to consumers, or multiple smaller capsules, which makes the product more expensive. Therefore, formulating nutritional or dietary supplements in extended release form is problematic and may not be cost effective.

Although the technology in the field of nutritional or dietary supplements does not offer the plethora of extended release delivery system that are available to drugs, there exists prior art extended release nutraceutical/dietary supplements, for example extended release drink technology. The extended release drink technology generally uses a water insoluble coating to slow down the release of the nutraceutical/dietary supplements. While this is a good way to control the release of active ingredients, it tends to give a chalky, gritty, or slimy mouthfeel and sediment when added to liquid. Further, such technologies are also provide low active payloads.

European Patent Application Publication No. 3242565 teaches a powder blend of (a) an immediate release powder and (b) extended release granule comprising a core. The core of the extended release granules comprises at least one performance enhancing component, one or more fatty materials, and optionally one or more swellable polymers such as microcrystalline cellulose and hydroxypropyl methyl cellulose. The core of the extended release granules is further coated with an ethyl cellulose-based coating or poly-vinyl based coating or hydrophobic shellac-based coating, or hydroxypropyl methyl cellulose based coating, as a barrier coating.

United States Patent Application Publication No. 2005181047 discloses a time or retarded release nutraceutical composition. The nutraceutical composition comprises pellets which are formed from an admixture of (i) a nutritional supplement; (ii) a saccharide such as refined sugar, monosaccharides, and disaccharides; (iii) an excipient such as silicon dioxide, microcrystalline cellulose, calcium phosphate and the like; (iv) a lubricant such as magnesium stearate, stearic acid, talc and the like; (v) an agglutinative such as hydroxypropyl methyl cellulose, ethyl cellulose, polyacrylates and the like; (vi) a stabilizing agent such as Shellac gum; and (vii) a plasticizer such as diethylphthalate, adipates, stearate and the like. Further, the pellet can also be present in the form of a core and a semipermeable coating surrounding that core wherein the core is comprised of the saccharide, the excipient, the lubricant and the agglutinate, and the coating is comprised of the lubricant, the stabilizing agent and the plasticizer.

PCT Publication No. WO2001035953 discloses a sustained release oral exercise and muscle enhancing composition of creatine and an insulin modulating agent such as arginine.

However, a need therefore exists for a suitable dosage form that has a flexibility to deliver a wide range of bio-active ingredients with varying physical properties and dosage/payload ranges, and also is capable of delivering relatively high dosage level of bio-active ingredients. Further, it would be desirable for such dosage forms could be manufactured with simple and conventional pharmaceutical techniques and materials.

### SUMMARY OF THE INVENTION

One aspect of the present disclosure provides a dispersible extended release granule composition comprising: (i) one or more bio-active ingredients; (ii) at least one water-soluble gel forming polymer; and (iii) at least one water-insoluble lipid material, wherein the bio-active ingredient and the water-soluble gel forming polymer are present in the form of dry, pre-granulated particles either partially or fully coated with the water-insoluble lipid material. In one non-limiting embodiment of the present disclosure, the bio-active ingredients is selected from the group consisting of essential amino acids, caffeine, and melatonin. In one non-limiting embodiment, the water-soluble gel forming polymer includes at least one polymer selected from the group consisting of hydroxypropyl methyl cellulose (HPMC), hydroxypropyl cellulose (HPC), carboxymethyl cellulose (CMC), guar-gum, acacia gum, and combinations thereof. In another non-limiting embodiment, the lipid material includes at least one material selected from the group consisting of palm oil, palm stearin, palm olein, coconut oil, medium chain triglycerides (MCT's), glyceryl mono caprylate, fatty acid esters, and combinations thereof.

In another non-limiting embodiment of the present disclosure, the dispersible extended release composition can further comprise a dispersant coating containing at least one dispersant which is partially or fully coated onto the surface of the dispersible extended release granules. The dispersant can be selected from the group consisting of lecithin, mono- and diglycerides, polysorbates, carrageenan, guar gum, and combinations thereof.

Another aspect of the present disclosure provides a process for preparing the dispersible extended release composition of the present disclosure wherein the process comprises the steps of: (i) granulating a dry blend of at least one bio-active ingredient and at least one water-soluble gel forming polymer in a fluid bed system using a spray granulation process to obtain pre-granulated particles of the dry blend of the bio-active ingredient and the water-soluble gel forming polymer; (ii) coating the pre-granulated particles obtained from the process step (i) with at least one water-insoluble lipid material in a fluid bed system using a spray granulation process to obtain the dispersible extended release granule composition. In another non-limiting embodiment of the present disclosure, the process further comprises a step of coating the surface of extended release granules with at least one dispersant in a fluid bed system using a spray granulation process.

### BRIEF DESCRIPTION OF THE FIGURES

Further embodiments of the present application can be understood with reference to the appended Figures.

FIG. 1(a), FIG. 1(b) and FIG. 1(c) illustrate the dissolution profile of essential amino acids (EAAs) tracking leucine, isoleucine and valine in the extended release granule composition of Example 1.

FIG. 2(a), FIG. 2(b) and FIG. 2(c) illustrate dissolution profile of essential amino acids tracking leucine, isoleucine, and valine in the compositions of Comparative Example 1 (CE. 1, CE. 2 and CE. 3).

FIG. 3(a) and FIG. 3(b) show dissolution profile of essential amino acids tracking leucine, isoleucine and valine in the compositions of Comparative Example 4 (CE. 4A and CE. 4B).

FIG. 4 shows dissolution profile of essential amino acids tracking leucine, isoleucine and valine in the composition of Comparative Example 5 (CE. 5).

FIG. 5 shows dissolution profile of caffeine present in the extended release granule composition of Example 2.

FIG. 6 shows dissolution profile of melatonin present in the extended release granule composition of Example 3.

### DETAILED DESCRIPTION OF THE INVENTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in many ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the present disclosure, the following terms, unless otherwise indicated, shall be understood to have the following meanings.

The use of the word “a” or “an” when used in conjunction with the term “comprising” may mean “one,” but it is also consistent with the meaning of “one or more,” “at least one.” and “one or more than one.” The use of the term “or” is used to mean “and/or” unless explicitly indicated to refer to alternatives only if the alternatives are mutually exclusive, although the disclosure supports a definition that refers to only alternatives and “and/or.” Throughout this application, the term “about” is used to indicate that a value includes the inherent variation of error for the quantifying device, the method(s) being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 1, 2, 3, 4, 5, 10, 15, 20, 30, 40, 50, 100, etc. The term “at least one” or “at least two” may extend up to 100 or 1000 or more depending on the term to which it is attached. In addition, the quantities of 100/1000 are not to be considered limiting as lower or higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first”, “second”, “third”, “fourth”, etc.) is solely for the purpose of differentiating between two or more items and, unless otherwise stated, is not meant to imply any sequence or order or importance to one item over another or any order of addition.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The terms “or combinations thereof” and “and/or combinations thereof” as used herein refer to all permutations and combinations of the listed items preceding the term. For example, “A, B. C, or combinations thereof” is intended to include at least one of: A, B, C, AB, AC, BC, or ABC and, if order is important in a particular context, also BA, CA, CB, CBA, BCA, ACB, BAC, or CAB. Continuing with this example, expressly included are combinations that contain repeats of one or more items or terms, such as BB, AAA, AAB, BBC, AAABCCCC, CBBAAA, CABABB, and so forth. The skilled artisan will understand that typically there is no limit on the number of items or terms in any combination, unless otherwise apparent from the context.

All percentages, ratio, and proportions used herein are based on a weight basis unless other specified.

As used herein, the term “bio-active ingredients” include physiologically or pharmacologically active substances intended for use in the treatment, prevention, diagnosis, cure or mitigation of disease or illness, or substances that provide some degree of nutritional or therapeutic benefit to animals or humans when consumed. The bio-active ingredients can include, but are not limited to, bio-active ingredients for pharmaceutical applications or for dietary or nutraceutical applications. Illustrative, but non-limiting examples of bio-active ingredients suitable for use for the purpose of the dispersible extended release granule composition of the present disclosure include hormones, proteins, amino-acids and amino acid derivatives, polypeptides, antigens, probiotic bacteria, prebiotics, enzymes, co-enzymes, cofactors, antioxidants, minerals, and mineral salts, vitamins, carbohydrates, phytochemicals, dextrose, phospholipids, other trace nutrients, oxygenators, brain-stimulating substances, energy provider, metabolic intermediates, botanical extracts, fatty acids, oat beta-glucan or other functional fibers, carnitine, bicarbonate, citrate, drugs, and other pharmaceutically, nutraceutically or therapeutically useful compounds.

As used herein, the term “extended release” can be described as a release of a bio-active ingredient(s) from a composition or dosage form over an extended period according to a desired profile to accomplish therapeutic or convenience objectives not offered by conventional dosage forms such as a solution or immediate release dosage form.

As used herein, the term “dispersible” means that the extended release granule composition of the present disclosure can be mixed with a suitable dispersion medium to form a macroscopically uniform mixture without the use of high shear mixing. The dispersion medium can be an aqueous medium or any other suitable liquid medium. Illustrative, but non-limiting examples of such suitable liquid medium include water, milk, juices, including fruit and vegetable juices, coconut water, alcoholic and non-alcoholic beverages and the like.

As used herein, the term “lipid materials” can be described as a member of a group of organic compounds that has lipophilic or amphipathic properties. Illustrative, but not limiting examples of the lipid materials suitable for the purpose of the present disclosure can include palm oil, fats, fatty oils, essential oils, waxes, steroids, sterols, phospholipids, glycolipids, sulpholipids, aminolipids, chromolipids, fatty acids or their esters and the like.

In one aspect, the present disclosure provides a dispersible extended release composition comprising: (i) one or more bio-active ingredient; (ii) at least one water soluble gel forming polymer; and (iii) at least one water insoluble lipid material. The extended release composition of the present disclosure is a dispersible granule composition, wherein the bio-active ingredient and the water-soluble gel forming polymers are present in the form of dry, pre-granulated particles. The pre-granulated particles are formed from a dry blend of the bio-active ingredients and the water-soluble gel forming polymers. The pre-granulated particles are further covered or coated with the water-insoluble lipid material. The water-insoluble lipid materials can either be partially or fully coated onto the surface of the pre-granulated particles of the extended release composition of the present disclosure.

The bio-active ingredients used in the extended release granule composition of the present disclosure can be bio-active ingredients suitable for pharmaceutical applications or for dietary or nutraceutical applications. In one non-limiting embodiment of the present disclosure, the bio-active ingredients can be a bio-active ingredient for nutraceutical or wellness applications. The bio-active ingredients for nutraceutical applications are also known as nutraceuticals or nutraceutical supplements in the related art. As used herein the term, “nutraceutical supplement” refers to a substance that exerts a physiological effect on an animal or human. The present dispersible extended release granule composition of the present disclosure can be suitable for a wide range of bio-active ingredients for nutraceutical applications. For example, the bio-active ingredients can be useful for applications related to energy boosting, managing sleep related disorders or improving sleep efficiency, joints and cartilage health as well as joint comfort and mobility, improving and maintaining the health conditions of primary organs such as digestive or GIT, bear, liver, pancreas, lungs, brain, bones, and muscles, relaxation and mood uplifting, cognitive health (nootropics), helping the body adapt to and resist physical, chemical, and environmental stress (adaptogens), weight management, sports nutrition, and the like.

Illustrative, but non-limiting examples of bio-active ingredients related to energy boosting include caffeine, guayusa, theophylline, theobromine, guarana, yerba mate extract, and the like. Illustrative, but non-limiting examples of bio-active ingredients suitable for treating sleep related disorders or improving sleep efficiency include melatonin, lemon balm extract, chamomile extract, valerian root extract, gamma amino butyric acid (GABA), and the like. Similarly, illustrative, but non-limiting examples of bio-active ingredients suitable for joint and cartilage health as well as joint comfort and mobility include glucosamine, chondroitin, methylsulfonylmethane (MSM), collagen, gelatin, and the like. Illustrative, but non-limiting examples of bio-active ingredients suitable for improving and maintaining digestive health include ginger extract, peppermint extract, licorice extract, bromelain, papain, and the like. Similarly, illustrative, but non-limiting examples of bio-active ingredients suitable for relaxation/mood uplifting include, L-theanine, S-adenosyl methionine (SAMe). Gingko biloba, and the like.

Illustrative, but non-limiting examples of bio-active ingredients suitable for cognitive health (nootropics) include choline, Ashwagandha, Theacrine, and the like. Further, illustrative, but non-limiting examples of bio-active ingredients for helping the body adapt to and resist physical, chemical, and environmental stress (adaptogens) include Ginseng extract, Rhodiola extract, Fenugreek extract, Tribulus terrestris extract, and the like. Illustrative, but non-limiting examples of bio-active ingredients for weight management include Epigallocatechin gallate (EGCG), L-arabinose, White kidney bean extract, Citrus aurantium extract, and the like. Illustrative, but non-limiting examples of bio-active ingredients for sports nutrition include amino acids such as L-citrulline. L-Arginine; Grape seed extract; Beet root extract and the like.

In one non-limiting embodiment of the present disclosure, the bio-active ingredient can include one or more essential or non-essential amino acids, caffeine and/or melatonin.

In one embodiment of the present disclosure, the bio-active ingredient can be one or more essential or non-essential amino acids. Amino acids are well known building blocks of proteins. These can also be used as an energy source by the body. Amino acids can be placed in three different groups: essential amino acids, non-essential amino acids and conditional amino acids. Essential amino acids cannot be produced by the body, and hence must come from the food, or dietary or nutraceutical supplements. Further, these amino acids are also very commonly used in sports nutrition for muscle repair and recovery as well as to fuel lean muscle development. In one non-limiting embodiment of the present disclosure, the amino acids can be one or more essential amino acids. Suitable examples of such amino acids include, but are not limited to, leucine, isoleucine, valine, L-lysine, histidine, phenylalanine, threonine, methionine, tryptophan, and combinations thereof. In another non-limiting embodiment of the present disclosure, the amino acids can be one or more non-essential amino acids. Suitable examples of such amino acids include, but are not limited to, L-citrulline, L-arginine, theanine, and the like.

In another embodiment of the present disclosure, the bio-active ingredient can be caffeine or its analogues. In yet another embodiment of the present disclosure, the bio-active ingredient can be melatonin or its analogues. The extended release composition according to the present disclosure can comprise a relatively high dosage/amount of the bio-active ingredient(s). The bio-active ingredient(s) can be present in an amount of at least 20 wt. %, based on the total weight of the composition. In one non-limiting embodiment of the present disclosure, the amount of bio-active ingredient can vary in the range of from 20 wt. % to 80 wt. %, based on the total weight of the composition. In one non-limiting embodiment of the present disclosure, other ranges of bio-active ingredient would include from 20 wt. % to 30 wt. %, from 30 wt. % to 40 wt. %, from 40 wt. % to 50 wt. %, from 50 wt. % to 60 wt. %, from 60 wt. % to 70 wt. %, or from 70 wt. % to 80 wt. %.

The water-soluble gel forming polymer present in the extended release composition of the present disclosure can include cellulose or cellulose ether polymers. Examples of the cellulose ether polymers can include but are not limited to, hydroxypropyl methyl cellulose (HPMC), hydroxypropyl cellulose (HPC), hydroxy ethyl cellulose, carboxymethyl cellulose (CMC) and combinations thereof. In one non-limiting embodiment, other polymers can include but not limited to guar gum, acacia gum and combinations thereof. In one non-limiting embodiment, the water-soluble gel forming polymer can be hydroxypropyl methyl cellulose (HPMC). The hydroxypropyl methyl cellulose can have a viscosity in the range of from about 4000 cP to about 200,000 cP, as measured according to the United States Pharmacopeia National Formulary (USP NF). In another embodiment, the viscosity of the hydroxypropyl methyl cellulose can vary in the range of from about 85,000 cP to about 200,000 cP, as measured according to the USP NF. The present disclosure contemplates use of relatively high molecular weight cellulosic polymers such as hydroxypropyl methyl celluloses, as these polymers swell and slow the release of the bio-active ingredients. Further, the water-soluble gel forming polymer can be present in an amount of from about 5 wt. % to about 90 wt. %, based on the total weight of the extended release composition. In another non-limiting embodiment of the present disclosure, the amount of water-soluble gel forming polymer varies in the range of from about 5 wt. % to about 10 wt. %, or from about 10 wt. % to about 20 wt. %, or from about 20 wt. % to about 30 wt. %, or from about 30 wt. % to about 40 wt. %, or from about 40 wt. % to about 50 wt. %, or from about 50 wt. % to about 60 wt. %, or from about 60 wt. % to about 70 wt. %, or from about 70 wt. % to about 80 wt. %, or from about 80 wt. % to about 90 wt. % based on the total weight of the extended release composition.

In one non-limiting embodiment, the water-insoluble lipid material can include, but is not limited to, palm oil, palm stearin, palm olein, coconut oil, medium chain triglycerides (MCT's), glyceryl mono caprylate, fatty acid esters such as vegetable stearic acid (VSA), and combinations thereof. The use of lipid coating onto the outer surface of the pre-granulating particles slows down the water up take and protects the particles from breaking down when mixed in a shaker bottler with a mixing ball. In one non-limiting embodiment of the present disclosure, the water-insoluble lipid material can be present in an amount of from about 2 wt. % to about 40 wt. % based on the total weight of the extended release composition. In another non-limiting embodiment, the water-insoluble lipid material can be present in an amount of from about 5 wt. % to about 35 wt. %, or from about 15 wt. % to 35 wt. %, or from about 10 wt. % to about 30 wt. %, or from 20 wt. % to about 30 wt. % based on the total weight of the extended release composition.

The extended release composition of the present disclosure can further comprise a dispersant coating comprising a dispersant. The use of a dispersant as the outermost coating on the surface of the extended release granules helps with dispersibility unlike other extended/controlled release ingredients that tend to clump when added to water. Illustrative, but non-limiting examples of such dispersants include, lecithin, mono- and diglycerides, polysorbates, carrageenan, guar gum, and combinations thereof. The dispersant can be present in an amount of from 1 wt. % to 5 wt. %, or from 1 wt. % to 2 wt. %, based on the total weight of the composition. In another non-limiting embodiment, it is contemplated to employ a dispersant in the outermost coating on the surface of the extended release granules.

The extended release granule composition according to the present disclosure can further comprise at least one additional ingredient(s) that includes, but is not limited to, ingredients selected from the categories of glidants, preservatives, flavors, sweeteners, colorants, binders and the like. The choice of such additional ingredients including their amounts are considered to be within the abilities of one skilled in the art. Further, it is specifically contemplated that these additional ingredients may be present in the pre-granulated form of the extended release granule composition. Alternatively, these additional ingredients may also be present in either one or both of the lipid and dispersant coatings.

Another aspect of the present disclosure provides a process for preparing the dispersible extended release composition of the present disclosure, particularly a process for preparing the dispersible extended release granule composition. The extended release granule composition of the present disclosure can be prepared using methods of granulating known in the related art. Such methods can include, but are not limited to, dry and wet granulation technology, including fluid bed granulation, high shear granulation, extrusion and spheronization, and spay drying. The process according to the present disclosure comprises the steps of (i) preparing a blend of at least one bio-active ingredient and at least one water-soluble gel forming polymer; (ii) granulating the blend obtained from the process step (i) to obtain pre-granulated particles of the blend; and (iii) coating the surface of the pre-granulated particles obtained from the process step (ii) with at least one water-insoluble lipid material to obtain dispersible extended release granule composition. The process can further comprise an optional step of coating the extended release granules of the present disclosure with a surface active, food grade dispersant. Illustrative examples of such dispersants can include, but are not limited to, lecithin, mono- and -diglycerides, polysorbates, carrageenan, guar gum and combinations thereof.

In a typical process step, the bio-active ingredient(s) and the water-soluble gel forming polymer(s) are blended to obtain a dry blend thereof. The obtained dry blend is then wet-granulated with water in a fluid bed system using a top spray granulation process to obtain pre-granulated particles of the dry blend comprising the bio-active ingredient and the water-soluble gel forming polymer. The pre-granulated particles are then dried and further coated with at least one water-insoluble lipid material to obtain dispersible extended the release granules composition. The coating of the water-insoluble lipid material can be carried out in the same fluid bed system using a spray granulation process. The dispersible extended release granule composition, thus obtained, can be further coated with at least one dispersant. The optional coating according to the present disclosure can be carried out in the same fluid bed system using a top spray granulation process.

The extended release granules obtained according to the process of the present disclosure can have mean particle size less than 300 μm. In one non-limiting embodiment of the present disclosure, the particle size of the extended release granule composition can vary from 200 μm to 300 μm.

The extended release granule composition according to the present disclosure is suitable for oral administration and can be formulated into suitable oral dosage forms. Examples of such oral dosage forms include, but are not limited to, dispersible powder, tablets, capsules, lozenges, stick packs and sachets. In one non-limiting embodiment of the present disclosure, the extended release granule composition is a dispersible powder. In another non-limiting embodiment of the present disclosure, the extended release granule composition is formulated into stick packs and sachets. Further, the extended release granule composition of the present disclosure, can be mixed with a dispersion medium to form an orally administrable extended release oral suspension. The forms of the extended release granule composition of the present disclosure, preferably in the forms of dispersible powder or stick packs or sachets, is easily dispersed in a dispersion medium. In another embodiment of the present disclosure, the dispersible extended release composition is an extended release oral suspension.

Further, the dispersible extended release granule composition of the present disclosure can be formulated in the form of different products. Suitable examples of such products include, but are not limited to, dispersible powered drink or powdered shake, powdered beverage products, liquid bioactive products, and the like.

In one non-limiting embodiment, the extended release granule composition of the present disclosure can be formulated into a pre and/or post workout powder shake. The pre and post workout powder shake can be used by athletes or healthy humans. In another non-limiting embodiment, the extended release granule composition of the present disclosure can be formulated into powdered alcoholic or non-alcoholic beverage products to deliver bio-active ingredients, preferably essential amino acids (EAAs) to prevent age related sarcopenia (muscle loss). In another non-limiting embodiment, the extended release granule composition of the present disclosure can be formulated into liquid bio-active products for pediatric, athletic, healthy human and geriatric populations.

While formulating the products, the extended release granule composition of the present disclosure can be dispersed in any suitable dispersion medium. The dispersion medium can be an aqueous medium or any other physiologically acceptable liquid medium. Suitable examples of such medium include, but are not limited to, milk, fruit juices, vegetable juices, coconut water, alcoholic or non-alcoholic medium, and other similar beverages. The dispersion of the extended release granule composition can be carried out with mechanical agitation in a shaker cup that includes a dispersion ball. In a non-limiting embodiment, the dispersion of the extended release granule composition in the suitable dispersion medium can be carried out manually without any mechanical agitation as in ready to disperse products.

The applicants of the present disclosure have surprisingly found that the extended release granule compositions of the present disclosure comprising relatively high loading levels of bio-active ingredients (in the range of from 20 wt. % to 80 wt. %, based on the total composition weight), survive intense agitation and release their payload in a controlled manner after dispersion over an extended period of time. While not being limited to the example of essential amino acids (EAAs), a formulation comprising a blend of essential amino acids can be released over a period of 4 to 6 hours in a USP dissolution apparatus with simulated gastric (pH 1.2) and intestinal fluid (pH 6.8) as an example. Further, with the specific combinations of water-soluble polymers as a part of the pre-granulated particles, and lipid and dispersant material coatings, the present application advantageously achieves an erosion and swelling based controlled release composition which does not clump on dispersion.

The following examples illustrate the present disclosure, parts and percentages being by weight, unless otherwise indicated. Each example is provided by way of explanation of the present disclosure, not limitation of the present disclosure. In fact, it will be apparent to those skilled in the art that various modifications and variations can be made in the present disclosure without departing from the scope or spirit of the invention. For instance, features illustrated or described as part of one embodiment, can be used on another embodiment to yield a still further embodiment. Thus, it is intended that the present disclosure covers such modifications and variations as come within the scope of the appended claims and their equivalents.

### EXAMPLES

### Example 1: Essential Amino Acids Containing Extended Release Granule Composition

### 1 (a) Preparation of Essential Amino Acids Blend:

A blend of essential amino acids (EAAs) was prepared by using the amino acids in weight proportions as listed in Table 1 below.

TABLE 1
Blend of Essential Amino Acids

| EAAs | Dosage (mg) | wt. % |
|---|---|---|
| Leucine | 2031.25 | 38.3 |
| Isoleucine | 1031.25 | 19.5 |
| Valine | 1031.25 | 19.5 |
| L-Lysine | 731.25 | 13.8 |
| Histidine | 131.25 | 2.5 |
| Phenylalanine | 131.25 | 2.5 |
| Threonine | 131.25 | 2.5 |
| Methionine | 81.25 | 1.8 |
| Total | 5300 | 100.0 |

### 1 (b) Preparation of Essential Amino Acids Extended Release Granule Composition:

The essential amino acid (EAAs) blend of Example 1(a) and hydroxypropyl methyl cellulose (HPMC) were mixed in a weight proportion listed in Table 2 below to obtain a dry blend thereof. The dry blend of EAAs-HPMC was then wet granulated with water in a fluid bed system using a top spray granulation process to obtain pre-granulated particles of EAA-HPMC blend. The obtained pre-granulated particles were dried and screened. In a separate step, Palm oil was gently heated until liquid and was then sprayed onto the pre-granulated particles of EAA-HPMC blend in the same fluid bed system using a top spray granulation process to obtain essential amino acids extended release granule composition. The amount of palm oil applied was sufficient to comprise 30 wt. % of the essential amino acids extended granule composition as shown in Table 2. Further, a top coat of lecithin was also applied on the surface of the essential amino acids extended granules using the same equipment and the top spray granulation process. The amount of lecithin applied was sufficient to constitute 2 wt. % of the essential amino acids extended release granule composition as shown in Table 2. After further drying and screening, the essential amino acids extended release granule composition was tested for dispersion and dissolution performance.

TABLE 2
EAAs Containing Extended Release Granule Composition

| | Weight | |
|---|---|---|
| Composition Components | percent (%) | Spatial orientation |
| Essential amino acid blend | 50.0 | Intragranular matrix |
| HPMC | 18.0 | Intragranular matrix |
| Palm oil | 30.0 | Extra granular coating |
| Lecithin | 2.0 | Extra granular coating |

### Dispersion and Dissolution Testing:

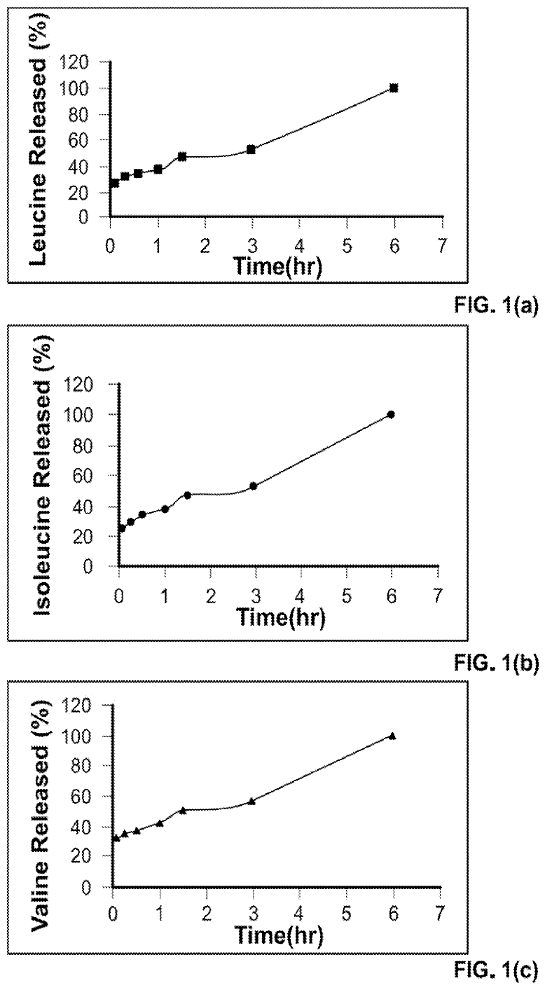

30 grams of the extended release granule composition of example 1(b) (equivalent to one serving size of EAAs of 15 gram) was dispersed in a shaker bottle with 300 ml of water and mixed with the shaker ball included in the bottle. The obtained mixture was vigorously shaken for 30 seconds and the reconstituted amino acid composition was then left standing for 10 minutes to simulate the variability on how consumers might use the drink. Dissolution testing was then conducted in a USP (United States Pharmacopoeia) apparatus I (paddle apparatus) in 600 ml of simulated gastric fluid (pH 1.2). The samples were taken at various time point and analyzed using high performance liquid chromatography (HPLC) technique. Dissolution results for Leucine, isoleucine and valine are shown in FIG. 1(a), FIG. 1(b), and FIG. 1(c) of the present disclosure, and it is clearly evident that these three amino acids (leucine, isoleucine and valine) showed biphasic release curve wherein 20-40% of the amino-acids is available immediately in the form of a loading dose while remaining amino-acids is extended released for up to 6 hours.

TABLE 3
Amount of Essential Amino Acids released from the
composition of Example I after 30 second shake

| Time (min) | Amount (%) |
|---|---|
| 5 mins | ~18 |
| 10 mins | ~23 |

### Example 2: Caffeine Containing Extended Release Granule Composition

The extended release granule composition comprising caffeine as a bio-active ingredient was prepared in the same manner as described above in Example 1. List of ingredients including their weight proportions used for preparing the caffeine containing extended release granule are shown below in Table 4.

TABLE 4
Caffeine Containing Extended Release Granule Composition

| | Weight | |
|---|---|---|
| Compositions Components | percent (%) | Spatial orientation |
| Caffeine | 73.0 | Intragranular matrix |
| HPMC | 9.0 | Intragranular matrix |
| Palm oil | 17.0 | Extra granular coating |
| Lecithin | 1.0 | Extra granular coating |

### Dispersion and Dissolution Testing:

The extended release granule composition comprising caffeine as the bio-active ingredient of Example 2 (equivalent to one serving size of caffeine of 300 mg) was dispersed in a shaker bottle with 300 ml of water and mixed with the shaker ball included in the bottle. The obtained mixture was vigorously shaken for 30 seconds and the reconstituted extended release caffeine composition was then left standing for 10 minutes to simulate the variability on how consumers might use the drink. Dissolution testing was then conducted in a USP (United States Pharmacopeia) apparatus I (paddle apparatus) in 600 ml. of DI water. The samples were taken at various time point and analyzed using high performance liquid chromatography (HPLC) technique. Dissolution profile of caffeine from the extended release composition of Example 2 is shown in FIG. 5 of the present disclosure and compared with the dissolution profile of pure caffeine. It is evident from the provided FIG. 5, about 40 wt. % to 60 wt. % (˜50 wt. %) of caffeine is released immediately in the form of a loading dose while the remaining caffeine is released for up to 6 hrs. This extended release of caffeine delivers the energy boost over an extended period of up to 6 hours with potentially fewer side effects like headaches, jitters, and “post-caffeine crash”.

### Example 3: Melatonin Containing Extended Release Granule Composition

The extended release granule composition comprising melatonin as a bio-active ingredient was prepared in the same manner as described above in Example 1. List of ingredients including their weight proportions used for preparing the melatonin containing extended release granule are shown below in Table 5. Two different extended release granule compositions having different wt. proportions of melatonin were prepared in this example: Example 3(A): with 72 wt. % of melatonin, and Example 3(B) with 53 wt. % of melatonin.

TABLE S
Melatonin Containing Extended Release Granule Composition

| | Example 3A | Example 3B | |
|---|---|---|---|
| Composition | Weight | Weight percent | |
| Components | percent (%) | (wt. %) | Spatial orientation |
| Melatonin | 72.0 | 53.0 | Intragranular matrix |
| HPMC | 9.0 | 17.0 | Intragranular matrix |
| Palm oil | 18.0 | 29.0 | Extra granular coating |
| Lecithin | 1.0 | 1.0 | Extra granular coating |

### Dispersion and Dissolution Testing:

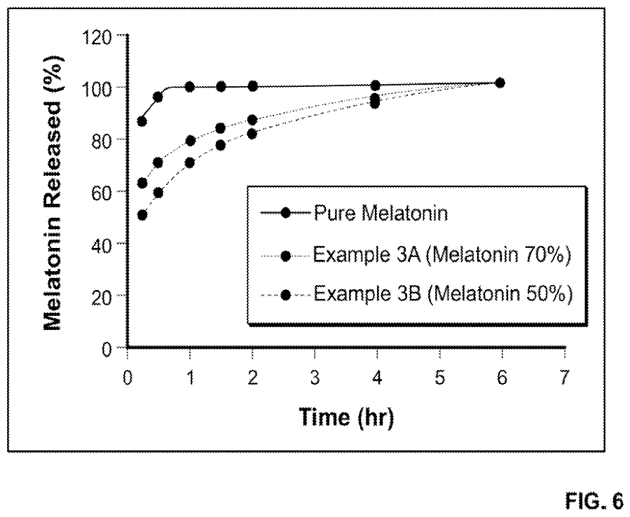

The melatonin extended release granule composition of Example 3 (equivalent to 100 mg of melatonin as one serving) was dispersed in a shaker bottle with 300 ml of water and mixed with the shaker ball included in the bottle. The obtained mixture was vigorously shaken for 30 seconds and the reconstituted melatonin containing extended release composition was then left standing for 10 minutes to simulate the variability on how consumers might use the drink. Dissolution testing was then conducted in a USP (United States Pharmacopeia) apparatus 1 (paddle apparatus) in 600 ml. of Deionized (DI) water. The samples were taken at various time point and analyzed using high performance liquid chromatography (HPLC) technique. Dissolution profile of Melatonin from the extended release granule of Example 3 is shown in FIG. 6 of the present disclosure and compared with the dissolution profile of pure Melatonin. As evident from the provided FIG. 6, 40 wt. % to 60 wt. % of melatonin is released immediately in the form of a loading dose while the remaining is released over the course of an evening for up to 6 hrs. This helps in falling asleep and maintain the sleep-wake cycle.

### Comparative Examples

### Comparative Examples 1, 2 and 3 (CE. 1. CE. 2, and CE. 3): Pre-Granulated Particles of Essential Amino Acids and Water-Soluble Gel Forming Polymers (without Lipid Coating)

In this example, pre-granulated particles of the essential amino acids and water-soluble gel forming polymers are prepared, which are not coated further with the water-insoluble lipid materials. In this example, three different types of pre-granulated particles were prepared using three different water-soluble gel forming polymers: guar gum, carboxymethyl cellulose (CMC) and hydroxypropyl methyl cellulose. In Comparative Example 1 (CE. 1), 85 gm. of active amino acids of example 1(a) and 15 gm. of guar gum (as a water-soluble gel polymer) were mixed together and dry blended, and pre-granulated as per the procedure of example 1(b) to obtain pre-granulated particles. These pre-granulated particles were not coated further with the water-insoluble lipid material. Two different types of pre-granulated particles were also prepared in the same manner using carboxymethyl cellulose (CMC) and hydroxypropyl methyl cellulose (HPMC) as the water-soluble gel forming polymers. (CE. 2 and CE. 3, respectively). These compositions were also not coated further with the water-insoluble lipid material.

TABLE 6
Pre-granulated Particles of Essential Amino Acids and Water-
Soluble Gel Forming Polymers (without lipid coating)

| Comparative Example | EAAs | CMC | Guar gum | HPMC |
|---|---|---|---|---|
| Compositions | (g) | (g) | (g) | (g) |
| CE. 1 | 85 | — | 15 | — |
| CE. 2 | 85 | 15 | — | — |
| CE. 3 | 85 | — | — | 15 |

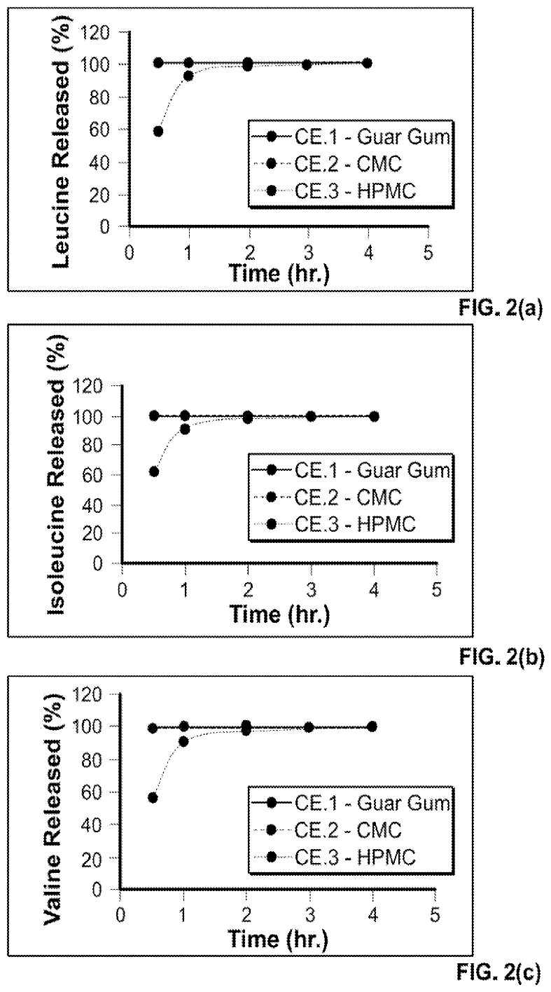

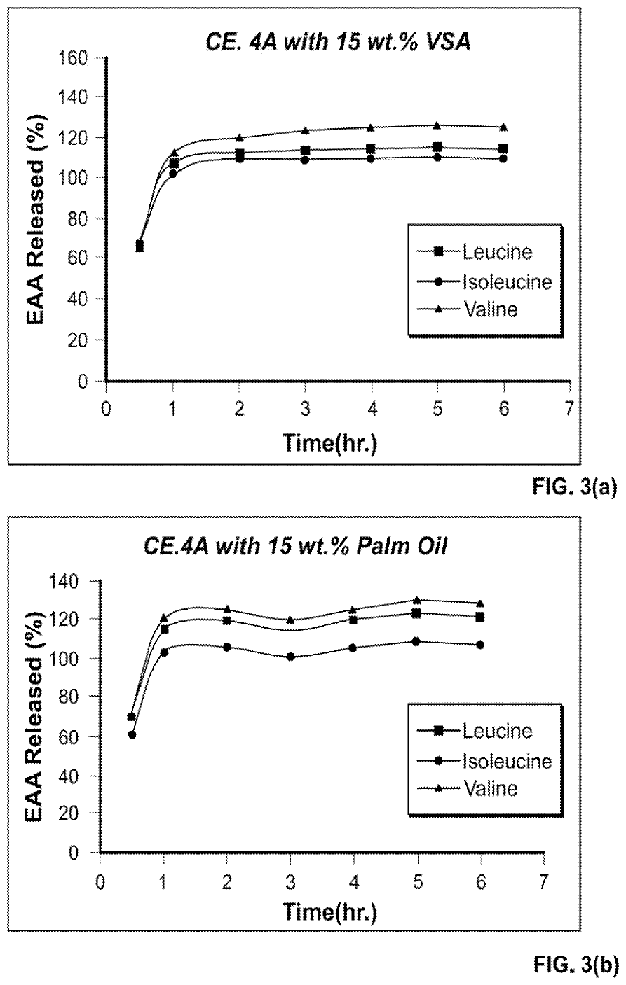

The dissolution test of the pre-granulated particles of the compositions of the Comparative Examples 1, 2 and 3 was carried out in the manner described above for the composition of Example 1, and illustrated in FIG. 2(a). FIG. 2(b) and FIG. 2(c) of the present disclosure. The dissolution test was carried out in a dissolution medium comprising 0.1N HCl (pH 1.2). USP (United States Pharmacopeia) Dissolution Apparatus 1 was used. As evident from the provided FIG. 2(a), FIG. 2(b) and FIG. 3(c), the pre-granulated particles of CE. 1 provided 1 hr. release profile with up to 60% EAAs released within the first 30 minutes. The absence of water-insoluble lipid coating resulted in inadequate release retardation of 2 hours or less. Further, only HPMC was found to sustain all three amino acids (Leucine, isoleucine and valine).

### Comparative Example 4: Essential Amino Acids Blend Coated with Only Water-Insoluble Lipid Material

### Comparative Example 4A (CE. 4A)

In two different examples, the pre-granulated particles of the Comparative Example 3 (CE. 3) were coated with vegetable stearic acid (VSA) (15%) and with palm oil (15%), respectively. The dispersion and dissolution profile of this composition was carried out in a dissolution medium comprising 0.1 N HCl (pH 1.2) and using USP (United States Pharmacopoeia) Dissolution Apparatus 1. The dissolution profile is shown in FIG. 3 of the present disclosure. It is clearly evident from the provided FIG. 3 that there is inadequate release retardation of EAAs due to insufficiently coating of granules with lipid coatings.

### Comparative Example 4B (CE. 4)

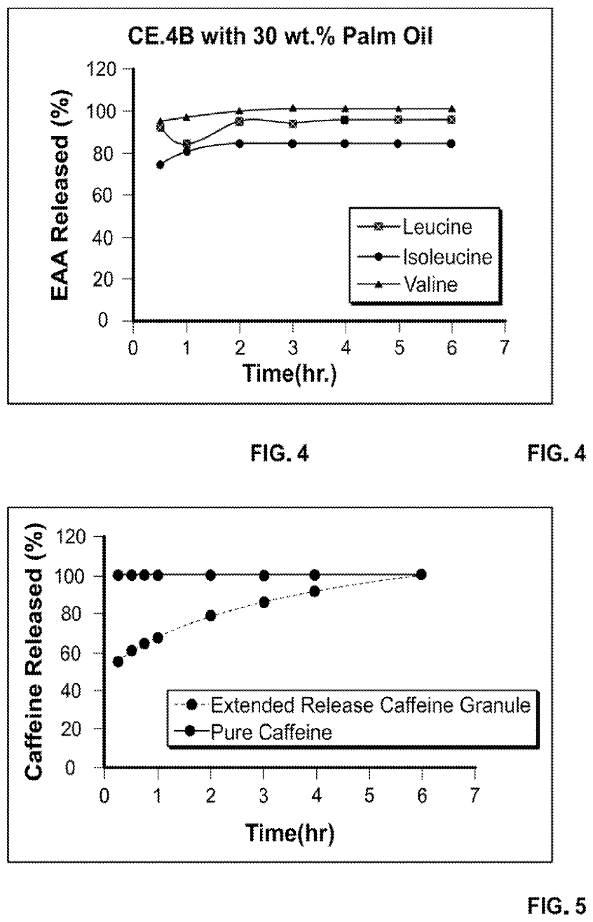

The physical blend of the essential amino acids were coated with only 30 wt. % palm oil. The dissolution profile test was carried out in a dissolution medium of 0.1 N HCl (pH 1.2) using USP (United States Pharmacopoeia) Dissolution Apparatus 1. The dissolution profile is illustrated in FIG. 4 of the present disclosure. It is clearly evident that without the use of the water-soluble gel forming polymers, the increased lipid coating of 30 wt. % was unable to restart EAAs release beyond 1 hour.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

What is claimed is:

1. A dispersible extended release granule composition comprising:
(i) one or more bio-active ingredients;
(ii) at least one water-soluble gel forming polymer; and
(iii) at least one water insoluble lipid material,
wherein the bio-active ingredient and the water-soluble gel forming polymer are present in the form of dry, pre-granulated particles either partially or fully coated with the water insoluble lipid material.

2. The composition of claim 1, wherein the bio-active ingredient is selected from the group consisting of pharmaceutical, dietary and nutraceutical actives.

3. The composition of claim 1, wherein the bio-active ingredient is selected from the group consisting of essential amino acids, caffeine, and melatonin.

4. The composition of claim 3, wherein the essential amino acids are selected from the group consisting of leucine, isoleucine, valine, L-lysine, histidine, phenylalanine, threonine, methionine, tryptophan, and combinations thereof.

5. The composition of claim 1, wherein the bio-active ingredient is present in an amount of at least 20 wt. % based on the total weight of the composition.

6. The composition of claim 1, wherein the bio-active ingredient is present in an amount of from 20 wt. % to 80 wt. % based on the total weight of the composition.

7. The composition of claim 1, wherein the water-soluble gel forming polymer includes at least one polymer selected from the group consisting of hydroxypropyl methyl cellulose, hydroxypropyl cellulose, carboxymethyl cellulose, guar-gum, acacia gum and combinations thereof.

8. The composition of claim 1, wherein the water-soluble gel forming polymer is hydroxypropyl methyl cellulose.

9. The composition of claim 1, wherein the hydroxypropyl methyl cellulose has a viscosity in the range of from 4000 cP to 200,000 cP measured according to the USP NF.

10. The composition of claim 1, wherein the water-soluble gel forming polymer is present in an amount of from 5 wt. % to 90 wt. % based on the total weight of the composition.

11. The composition of claim 1, wherein the water insoluble lipid material includes at least one material selected from the group consisting of palm oil, palm stearin, palm olein, coconut oil, medium chain triglycerides (MCT's), glyceryl mono caprylate, fatty acid esters, and combinations thereof.

12. The composition of claim 1, wherein the water insoluble lipid material is present in an amount of from 2 wt. % to 40 wt. % based on the total weight of the composition.

13. The composition of claim 1, wherein the water insoluble lipid material is present in an amount of from 15 wt. % to 35 wt. % based on the total weight of the composition.

14. The composition of claim 1, further comprising a dispersant coating comprising a dispersant which is partially or fully coated onto the surface of the dispersible extended release granules.

15. The composition of claim 14, wherein the dispersant is selected from the group consisting of lecithin, mono- and diglycerides, polysorbates, carrageenan, guar gum and combinations thereof.

16. The composition of claim 15, wherein the dispersant is present in an amount of from 1 wt. % to 5 wt. % based on the total weight of the composition.

17. The composition of claim 1 formulated as an oral dosage form.

18. The composition of claim 17, wherein the oral dosage form is selected from the group consisting of dispersible powders, tablets, capsules, lozenges, stick packs and sachets.

19. The composition of claim 18, wherein the oral dosage form in dispersible powder, stick packs or sachets form is dispersible in an aqueous medium or other physiologically acceptable liquid mediums selected from the group consisting of milk, fruit juices, vegetable juices, alcoholic and non-alcoholic beverages.

20. The composition of claim 19, wherein the extended release composition is an extended release oral suspension.

21. The composition of claim 1 formulated into a pre and/or post workout powder shake for athletes or healthy humans.

22. The composition of claim 1 formulated into powdered beverage products to deliver at least one bio-active ingredient to prevent age related sarcopenia (muscle loss), wherein the bio-active ingredient comprises essential amino acids.

23. The composition of claim 1 formulated as liquid bioactive products for pediatric, athletic, healthy human and geriatric populations.

24. A process for preparing the dispersible extended release granule composition of claim 1 comprising the steps of: (i) granulating a dry blend of at least one bio-active ingredient and at least one water soluble gel forming polymer in a fluid bed system using a spray granulation process to obtain pre-granulated particles of the dry blend of the bio-active ingredient and the water soluble gel forming polymer: (ii) coating the pre-granulated particles obtained from the process step (i) with at least one water insoluble lipid material in a fluid bed system using a spray granulation process to obtain the dispersible extended release composition in the form of granules.

25. The process of claim 24 further comprising coating the surface of extended release granules with at least one dispersant in a fluid bed system using a spray granulation process.
